(19) United States
(12) Patent Application Publication
(10) Pub. No.: US 2026/0068892 A1
(43) Pub. Date: Mar. 12, 2026

(54) RNA INTERFERENCE (RNAI) FOR CONTROL OF JAPANESE BEETLE

(71) Applicant: University of Kentucky Research Foundation, Lexington, KY (US)

(72) Inventors: Subba Reddy Palli, Lexington, KY (US); Ramesh Dhandapani, Lexington, KY (US)

(21) Appl. No.: 19/124,751

(22) PCT Filed: Nov. 9, 2023
(86) PCT No.: PCT/US2023/079268

§ 371 Date: Apr. 25, 2025

Related U.S. Application Data
(60) Provisional application No. 63/423,851, filed on Nov. 9, 2022.

(51) Int. Cl. A01N 63/60; A01N 63/23; A01P 7/04; C12N 15/113

(52) U.S. Cl. CPC A01N 63/60; A01N 63/23; A01P 7/04; C12N 15/113; C12N 2310/14; C12N 2310/53

(57) ABSTRACT

The presently disclosed subject matter includes a composition comprising inactivated bacteria containing dsRNA targeting a lethal gene of an insect through RNA interference (RNAi) mechanism. The presently disclosed subject matter also includes a method of inducing RNAi in an insect by administering to the insect a composition comprising an inactivated bacteria containing expressed dsRNA targeting a lethal gene of the insect. The presently disclosed subject matter also includes a method of protecting a plant from an insect, comprising applying to the plant a composition comprising an inactivated bacteria containing expressed dsRNA targeting RNAi a lethal gene of the insect.

### RELATED APPLICATIONS

This application claims priority from U.S. Provisional Application Ser. No. 63/423,851, filed Nov. 9, 2022, the entire disclosure of which is incorporated herein by this reference.

### TECHNICAL FIELD

The presently disclosed subject matter relates to a composition comprising inactivated bacteria containing dsRNA for targeting a lethal gene of an insect through RNA interference (RNAi) mechanism. The presently disclosed subject matter also relates to a method of inducing RNAi in an insect by administering to the insect a composition comprising an inactivated bacteria containing expressed dsRNA targeting a lethal gene of the insect. The presently disclosed subject matter also relates to a method of protecting a plant from an insect, comprising applying to the plant a composition comprising an inactivated bacteria containing expressed dsRNA targeting RNAi a lethal gene of the insect.

### INTRODUCTION

Japanese beetles (Popilia japonica) are a significant problem for plants, especially in North America, where they are considered invasive. In their introduced range, Japanese beetles have fewer natural predators and parasites to keep their populations in check, which allows them to thrive and reproduce unchecked.

Currently, harm caused by Japanese beetles is particularly notable in the eastern portion of the United States, and preventing its spread to western states is a high priority. The main routes of spread of this pest to other regions of the country are thought to be through cargo transport by airplanes and plants produced in nurseries. Considerable efforts, including the use of chemicals insecticides, building exclusion facilities, and employing people to inspect each carrier, are currently undertaken at airports and cargo transportation facilities in areas with established populations of P. japonica.

Japanese beetles are voracious leaf-eating insects that consume the soft tissue between leaf veins, leaving behind a skeletonized, damaged leaf. They feed on over 300 plant species, including field crops, ornamental plants, fruit trees, vegetables, and turf grass. Their feeding can significantly defoliate plants and reduce crop yields.

Japanese beetles cause substantial economic losses in agriculture and horticulture. Feeding by Japanese beetles weakens plants and makes them more susceptible to diseases and other pests. This can further reduce the overall health and vigor of affected plants. Crop damage, reduced fruit production, and the cost of implementing control measures all contribute to economic losses for farmers and gardeners. In addition to economic and agricultural concerns, Japanese beetles also cause aesthetic damage to ornamental plants, which can be particularly frustrating for homeowners and gardeners.

Over time, Japanese beetles have developed resistance to many chemical pesticides, making it challenging to control their populations with traditional chemical methods. The use of chemical pesticides can have environmental implications, including the potential harm to non-target species, water contamination, and the development of pesticide-resistant pests. Therefore, more sustainable and environmentally friendly pest control strategies are needed.

Improved pest control strategies are essential to mitigate the damage caused by Japanese beetles. Beneficial approaches would address feeding damage, plant stress, and economic impact caused from damage due to Japanese beetles, and would additionally reduce reliance on chemical treatments and minimize environmental impact.

Accordingly, there remains a need in the art for improved methods and compositions for controlling Japanese beetles.

### SUMMARY

The presently-disclosed subject matter meets some or all of the above-identified needs, as will become evident to those of ordinary skill in the art after a study of information provided in this document.

RNA interference (RNAi) has emerged as a powerful tool for functional genomic studies, as well as a potential method for pest management. The presently-disclosed subject matter provides unique compositions and methods for cost-effective and enhanced delivery of double-stranded RNA (dsRNA) to the target insects, which are important for extensive applications needed for pest control and protection of plants from damage created by insects.

The presently-disclosed subject matter makes use of genetically engineered bacterial expression of dsRNA targeting the one or more lethal genes of a target insect. The bacteria is then in activated, and provided as a composition containing the dsRNA for use in insect control. For example, Actin is a lethal gene of Japanese beetles. Bacteria expressing ds Actin can be inactivated (e.g., heat-killed) and provided in a composition that will contain dsActin.

The composition can be administered to the target Japanese beetles by applying it to a plant or group of plants for protection. The composition can also be administered to the target Japanese beetles by applying it to plants near areas such as airports, transportation facilities and nurseries. In this manner, the Japanese beetle ingests the composition.

Upon ingestion of the dsRNA targeting a lethal gene, reduced expression of that gene occurs, and consequently, mortality in the dsRNA-fed insects.

In some embodiments, the presently disclosed subject matter includes a composition comprising inactivated bacteria containing dsRNA for a lethal gene of an insect through RNA interference (RNAi) mechanism. In some embodiments, the presently disclosed subject matter also includes a method of inducing RNAi in an insect by administering to the insect a composition comprising an inactivated bacteria containing expressed dsRNA targeting a lethal gene of the insect. In some embodiments, the presently disclosed subject matter also includes a method of protecting a plant from an insect, comprising applying to the plant a composition comprising an inactivated bacteria containing expressed dsRNA targeting RNAi a lethal gene of the insect.

In some embodiments, the presently disclosed subject matter includes a composition comprising a nanoparticle containing dsRNA for a lethal gene of an insect through RNA interference (RNAi) mechanism. In some embodiments, the presently disclosed subject matter also includes a method of inducing RNAi in an insect by administering to the insect a composition comprising an inactivated bacteria containing expressed dsRNA targeting a lethal gene of the insect. In some embodiments, the presently disclosed subject matter also includes a method of protecting a plant from an insect, comprising applying to the plant a composition comprising an inactivated bacteria containing expressed dsRNA targeting RNAi a lethal gene of the insect.

Certain aspects of the presently-disclosed subject matter are based in part on the discovery that use of free or naked dsRNA is ineffective; however, if the dsRNA expressed and contained in an inactivated bacteria, or the dsRNA is contained in a nanoparticle, efficacy is achieved.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned: likewise, those features can be applied to other embodiments of the presently-disclosed subject matter, whether listed in this Summary or not. To avoid excessive repetition, this Summary does not list or suggest all possible combinations of such features.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1. Bacterially expressed dsRNA: dsRNA corresponding to GFP and JB-Actin (100 ml culture) induced by IPTG in the bacterial strain HT115 (DE3). Bacterial total RNA was extracted from 1 ml culture and run 1% agarose gel electrophoresis.

FIGS. 2A and 2B. RNAi induced by heat-killed bacteria expressing dsActin in Japanese beetle. Bacterial culture (100 ml) was centrifuged 2500 rpm for 10 mins at 4° C. The supernatant was discarded and the pellet was washed with 1λPBS buffer. The pellet was dissolved by 2 ml double distilled water and incubated at 80° C. for 30 mins and 2 ml of inactivated bacteria was sprayed on (FIG. 2A) Linden and (FIG. 2B) Rose leaves; mortality was recorded on 6th-day post-feeding and percent mortality was recorded. The experiment was repeated three times under the same conditions. (Mean±S.D) (N=30).

FIG. 3. RNAi induced by heat-killed bacteria in Japanese beetle by feeding bioassay. The gene expression level was confirmed three days after fed bacteria by RT-qPCR analysis. The relative mRNA levels were normalized using GAPDH as a reference gene. Mean±S.E (N=5) are shown. Asterisk shows a statistical difference (P<0.05).

FIG. 4. Mortality of beetles 10 days after start of feeding on leaves collected from plants in the field sprayed with dsActin or dsGFP.

FIG. 5. Mortality of beetles fed on linden leaves treated with dsGFP (control), dsGFP plus adjuvant (ad) (Control), dsActin, dsActin plus adjuvant (Ad) and Carbaryl (positive control). The beetles alive and feeding were counted on each day after treatment and the percent mortality was calculated. Mean and standard deviation of two duplicate experiments are shown.

FIG. 6. Mortality of beetles fed on rose leaves treated with dsGFP (control), dsGFP plus adjuvant (ad) (Control), dsActin, dsActin plus adjuvant (Ad) and Carbaryl (positive control). The beetles alive and feeding were counted on each day after treatment and the percent mortality was calculated. Mean and standard deviation of two duplicate experiments are shown.

FIG. 7. Okra leaves collected at 24, 48, 72, 96 and 168 hr after spraying dsGFP or dsActin were collected and fed to beetles. The mortality of beetles was recorded daily until 10 days after treatment.

FIG. 8. Number of beetles detected weekly for seven weeks after spraying dsGFP (control) or dsActin on Okra plants. The number of beetle counts on 10 plants per treatment are shown.

FIG. 9. Leaf damage by beetles recorded weekly for seven weeks after spraying dsGFP (control) or dsActin on Okra plants. The percent leaf damage estimates on 10 plants per treatment are shown.

FIG. 10. Okra yield quantified at the end of the season from 10 plants from dsGFP and dsActin treatments.

FIG. 11. Number of beetles detected weekly for seven weeks after spraying dsGFP (control) or dsActin on Okra plants. The number of beetle counts on 10 plants per treatment are shown.

FIG. 12. Leaf damage by beetles recorded weekly for seven weeks after spraying dsGFP (control) or dsActin on Okra plants. The percent leaf damage estimates on 10 plants per treatment are shown.

FIG. 13A-13C. Stability of dsRNA in Japanese beetle lumen content. One microgram of dsRNA was exposed to 10 μg of midgut lumen contents and incubated 0, 1, 3, 6, 12 and 24 hours in room temperature. The sample mixtures were collected and analyzed in 1% agarose gel electrophoresis.

FIG. 14. Identification of RNAi target genes in Japanese beetle. Ten micrograms of dsRNA targeting each gene was injected into each adult beetle. Mortality was scored on the 10^{th} day after injection of dsRNA. dsGFP was used as a control. The mortality rate for the three genes that showed the greatest efficiency was used in our screening assay.

FIG. 15. Feeding naked dsRNA by linden leaf in Japanese beetle. A total of 10 μg/beetle, 15 μg/beetle, 100 μg/10 beetle and 150 μg/10 beetle of dsGFP and dsActin were fed to adult beetles for 3 days. dsRNA was spread on 1 cm diameter leaf discs for individual beetles and 4 cm×4 cm leaf squares in group assays. The mortality was observed up to 10 days after feeding and percent mortality was recorded. There was no significant mortality was observed in this assay (N=3).

FIG. 16. Nanoparticle mediated RNAi in Japanese beetle by leaf feeding bioassay. PLL:EGCG:dsRNA nanoparticles were added to a 4 cm×4 cm square of linden leaf and adult beetles were allowed to feed (total 10 μg/beetle. 100 μg/10 beetle & 150 μg/10 beetle of dsGFP and dsActin) for 3 days. The mortality was observed up to 10 days post feeding and percent mortality was recorded. (N=3).

FIGS. 17A and 17B. RNAi induced by heat-killed bacteria expressing dsActin in Japanese beetle. Heat inactivated bacteria expressing dsGFP or dsActin were sprayed on (A) linden leaves and (B) Rose he mortality was recorded on 6^{th} day post-feeding and percent mortality was recorded. (Mean±S.D) (N=30).

FIGS. 18A and 18B. Feeding of dsRNA after knockdown of dsRNase's in Japanese beetle. A total of 40 ug of mixed dsRNase's (1, 2, 3 & 4) were injected into each adult Japanese beetle. After three days, fifteen micrograms of dsGFP and dsActin were fed to the beetles for up to three days. The mortality was scored up to 10 days post-feeding. There was no significant mortality was observed in this assay.

FIG. 19. Feeding bioassay with Japanese beetle exposed to Bacillus thuringiensis toxin (Bt) and Bt-dsRNA. A total of 150 μg/10 beetle of Bt-dsGFP and Bt-dsActin was added to a linden leaf and beetles were allowed to feed up to three days. After three days mortality was scored up to 10 days post-feeding

FIG. 20. Feeding bioassay with Japanese beetle exposed to Bacillus thuringiensis toxin (Bt) and Bt/NP-dsRNA. A total of 150 μg/10 beetle of Bt/NP-dsGFP and Bt/NP-dsActin was added to a linden leaf and beetles were allowed to feed up to three days. After three days mortality was scored up to 10 days post-feeding.

### REFERENCE TO AN ELECTRONIC SEQUENCE LISTING

The contents of the electronic sequence listing (Palli_2526WO_Sequence_Listing.xml; Size: 88 KB; and Date of Creation: 11/9/2023) is herein incorporated by reference in its entirety.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes a composition comprising inactivated bacteria containing dsRNA targeting a lethal gene of an insect through RNA interference (RNAi) mechanism. As disclosed herein, the bacteria is inactivated after the desired dsRNA has been expressed in the bacteria. Inactivation can occur using various methods known in the art, for example, the bacteria can be heat-killed or subjected to ultraviolet radiation. In some embodiments, the composition further includes Bacillus thuringiensis toxin (Bt).

In some embodiments, the composition also includes an adjuvant. As will be recognized by those of ordinary skill in the art, adjuvants for use with insect control composition are known in the art. In some embodiments, the adjuvant is provided to increase the ability of the composition to adhere to plant surfaces, penetrate insect cuticles, or spread evenly across the plant. This can lead to better insect control and pest management. In some embodiments, the adjuvants act as wetting agents or surfactants, helping the insecticide to spread and stick to the plant's surfaces, ensuring thorough coverage of the target areas. In some embodiments, the adjuvants can act as spreaders, helping the insecticide to disperse more evenly, and as stickers, improving its adhesion to plant surfaces. This ensures that the insect control composition remains effective for a longer period. In some embodiments, the adjuvants can reduce the risk of drift, which can occur when insecticides are applied as sprays. By minimizing drift, they help ensure that the pesticide is directed at the target area and does not harm non-target plants or areas.

In some embodiments of the composition, the lethal gene of the insect is a lethal gene of an insect is of the order Coleoptera. In some embodiments of the composition, the lethal gene of the insect is a lethal gene of an insect is of the genus Popillia. In some embodiments of the composition, the lethal gene of the insect is a lethal gene of an insect is of the species Popillia japonica. In some embodiments of the composition, the lethal gene is selected from the group consisting of Cactus, asnap, sinra, hsp, gw, srp, rop, pp1a, rpn7, rpt3, Mesh, HEL25E, Sec23, SAR1, SSK, Sam-S, VhaSFD, Sec61α, Nito, snRNP, Rpn11, Rpn12, Ebony, Surf4, Prosα1, Prosα6, Uba1, Chc, Shi, ATPsvn-β, Cas, Prosβ5, RpL6, Main, unc-104, DSP1. Fkh, Hel25E, Gcm, spt16, NCM, ROP, RPB7, DRE4, RP11140, snf7, Sac1, actin, Iap, and combinations thereof.

The presently disclosed subject matter also includes a method of inducing RNAi in an insect by administering to the insect a composition comprising an inactivated bacteria containing expressed dsRNA targeting a lethal gene of the insect. In some embodiments, the composition further includes Bacillus thuringiensistoxin (Bt).

In some embodiments of the method, administration is oral, such that the insect ingests the composition.

In some embodiments, the method also involves depositing the composition on a surface in an environment of the insect. In some embodiments, the surface is a plant. In this regard, the composition can be deposited on the leaves or petals of a plant. In some embodiments, the composition can be applied to a plant or group of plants for protection. In some embodiments, the composition can also be administered to the target insects in areas susceptible to inadvertently transporting that insect, by applying it near areas such as airports, transportation facilities, and nurseries.

In some embodiments, the method further involves re-depositing the composition on the surface every 1, 2, 3, 4, 5, 6, or 7 days.

In some embodiments of the method, the lethal gene of the insect is a lethal gene of an insect is of the order Coleoptera. In some embodiments of the method, the lethal gene of the insect is a lethal gene of an insect is of the genus Popillia. In some embodiments of the method, the lethal gene of the insect is a lethal gene of an insect is of the species Popillia japonica. In some embodiments of the method, the lethal gene is selected from the group consisting of Cactus, asnap, sinra, hsp, gw, srp, rop, pp1a, rpn7, rpt3, Mesh, HEL25E, Sec23, SAR1, SSK, Sam-S, VhaSFD, Sec61α, Nito, snRNP, Rpn11, Rpn12, Ebony, Surf4, Prosα1, Prosα6, Uba1, Chc, Shi, ATPsyn-β, Cas, Prosβ5, RpL6, Mam, unc-104, DSP1, Fkh, Hel25E, Gcm, spt16, NCM, ROP, RPB7, DRE4, RP11140, snf7, Sac1, actin, Iap, and combinations thereof.

The presently disclosed subject matter also includes a method of protecting a plant from an insect, comprising applying to the plant a composition comprising an inactivated bacteria containing expressed dsRNA targeting RNAi a lethal gene of the insect.

In some embodiments, the composition further includes Bacillus thuringiensis toxin (Bt). In some embodiments, the method further involves detecting synergistic protection of the plant, as compared to use of dsRNA or Bt alone.

In some embodiments, the method further involves re-depositing the composition on the surface every 1, 2, 3, 4, 5, 6, or 7 days.

In some embodiments of the method, the lethal gene of the insect is a lethal gene of an insect is of the order Coleoptera. In some embodiments of the method, the lethal gene of the insect is a lethal gene of an insect is of the genus Popillia. In some embodiments of the method, the lethal gene of the insect is a lethal gene of an insect is of the species Popillia japonica. In some embodiments of the method, the lethal gene is selected from the group consisting of Cactus, asnap, sinra, hsp, gw, srp, rop, pp1a, rpn7, rpt3, Mesh, HEL25E, Sec23, SAR1, SSK, Sam-S, VhaSFD, Sec61α, Nito, snRNP, Rpn11, Rpn12, Ebony, Surf4, Prosα1, Prosα6, Uba1, Chc, Shi, ATPsyn-β, Cas, Prosβ5, RpL6, Main, unc-104, DSP1, Fkh, Hel25E, Gcm, spt16, NCM, ROP, RPB7, DRE4, RP11140, snf7, Sac1, actin, Iap, and combinations thereof.

The presently-disclosed subject matter includes a composition comprising a nanoparticle containing dsRNA targeting a lethal gene of an insect through RNA interference (RNAi) mechanism. As disclosed herein, the nanoparticle could be, for example, a nanoparticle as described in U.S. Pat. No. 11,571,394 for modified-RNA nanoparticles for induction of RNA interference. In some embodiments, the composition further includes Bacillus thuringiensis toxin (Bt).

In some embodiments, the composition also includes an adjuvant. As will be recognized by those of ordinary skill in the art, adjuvants for use with insect control composition are known in the art. In some embodiments, the adjuvant is provided to increase the ability of the composition to adhere to plant surfaces, penetrate insect cuticles, or spread evenly across the plant. This can lead to better insect control and pest management. In some embodiments, the adjuvants act as wetting agents or surfactants, helping the insecticide to spread and stick to the plant's surfaces, ensuring thorough coverage of the target areas. In some embodiments, the adjuvants can act as spreaders, helping the insecticide to disperse more evenly, and as stickers, improving its adhesion to plant surfaces. This ensures that the insect control composition remains effective for a longer period. In some embodiments, the adjuvants can reduce the risk of drift, which can occur when insecticides are applied as sprays. By minimizing drift, they help ensure that the pesticide is directed at the target area and does not harm non-target plants or areas.

In some embodiments of the composition, the lethal gene of the insect is a lethal gene of an insect is of the order Coleoptera. In some embodiments of the composition, the lethal gene of the insect is a lethal gene of an insect is of the genus Popillia. In some embodiments of the composition, the lethal gene of the insect is a lethal gene of an insect is of the species Popillia japonica. In some embodiments of the composition, the lethal gene is selected from the group consisting of Cactus, asnap, sinra, hsp, gw, srp, rop, pp1a, rpn7, rpt3, Mesh, HEL25E, Sec23, SAR1, SSK, Sam-S, VhaSFD, Sec61α, Nito, snRNP, Rpn11, Rpn12, Ebony, Surf4, Prosα1, Prosα6, Uba1, Chc, Shi, ATPsyn-β, Cas, Prosβ5, RpL6, Main, unc-104, DSP1, Fkh, Hel25E, Gcm, spt16, NCM, ROP, RPB7, DRE4, RP11140, snf7, Sac1, actin, Iap, and combinations thereof.

The presently disclosed subject matter also includes a method of inducing RNAi in an insect by administering to the insect a composition comprising a nanoparticle containing expressed dsRNA targeting a lethal gene of the insect. In some embodiments, the composition further includes Bacillus thurengensis toxin (Bt).

In some embodiments of the method, administration is oral, such that the insect ingests the composition.

In some embodiments, the method also involves depositing the composition on a surface in an environment of the insect. In some embodiments, the surface is a plant. In this regard, the composition can be deposited on the leaves or petals of a plant. In some embodiments, the composition can be applied to a plant or group of plants for protection. In some embodiments, the composition can also be administered to the target insects in areas susceptible to inadvertently transporting that insect, by applying it near areas such as airports, transportation facilities, and nurseries.

In some embodiments, the method further involves re-depositing the composition on the surface every 1, 2, 3, 4, 5, 6, or 7 days.

In some embodiments of the method, the lethal gene of the insect is a lethal gene of an insect is of the order Coleoptera. In some embodiments of the method, the lethal gene of the insect is a lethal gene of an insect is of the genus Popillia. In some embodiments of the method, the lethal gene of the insect is a lethal gene of an insect is of the species Popillia japonica. In some embodiments of the method, the lethal gene is selected from the group consisting of Cactus, asnap, sinra, hsp, gw, srp, rop, pp1a, rpn7, rpt3, Mesh, HEL25E, Sec23, SAR1, SSK, Sam-S, VhaSFD, Sec61α, Nito, snRNP, Rpn11, Rpn12, Ebony, Surf4, Prosα1, Prosα6, Uba1, Chc, Shi, ATPsyn-β, Cas, Prosβ5, RpL6, Main, unc-104, DSP1, Fkh, Hel25E, Gcm, spt16, NCM, ROP, RPB7. DRE4, RP11140, snf7, Sac1, actin, Iap, and combinations thereof.

The presently disclosed subject matter also includes a method of protecting a plant from an insect, comprising applying to the plant a composition comprising a nanoparticle containing expressed dsRNA targeting RNAi a lethal gene of the insect.

In some embodiments, the composition further includes Bacillus thuringiensis toxin (Bt). In some embodiments, the method further involves detecting synergistic protection of the plant, as compared to use of dsRNA or Bt alone.

In some embodiments, the method further involves re-depositing the composition on the surface every 1, 2, 3, 4, 5, 6, or 7 days.

In some embodiments of the method, the lethal gene of the insect is a lethal gene of an insect is of the order Coleoptera. In some embodiments of the method, the lethal gene of the insect is a lethal gene of an insect is of the genus Popillia. In some embodiments of the method, the lethal gene of the insect is a lethal gene of an insect is of the species Popillia japonica. In some embodiments of the method, the lethal gene is selected from the group consisting of Cactus, asnap, sinra, hsp, gw, srp, rop, pp1a, rpn7, rpt3, Mesh, HEL25E, Sec23, SAR1, SSK, Sam-S, VhaSFD, Sec61α, Nito, snRNP, Rpn11, Rpn12, Ebony, Surf4, Prosα1, Prosα6, Uba1, Chc, Shi, ATPsyn-β, Cas, Prosβ5, RpL6, Mam, unc-104, DSP1, Fkh, Hel25E, Gcm, spt16, NCM, ROP, RPB7, DRE4, RP11140, snf7, Sac1, actin, Iap, and combinations thereof.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases, such as GENBANK® and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “administering” and “administration” refer to any method of providing a pharmaceutical preparation to a subject. Such methods are well known to those skilled in the art and include, but are not limited to, oral administration, dermal administration via the cuticle, ophthalmic administration, intracerebral administration, and respiratory administration, and injectable administration. Administration can be continuous or intermittent.

As used herein, the term “lethal genes” refer to genes which are known in the art to be lethal if knocked down or knocked out in an insect. Lethal genes may refer to the genes: cactus, asnap, sinra, hsp, gw, srp, rop, pp1a, rpn7, rpt3, Mesh. HEL25E, Sec23. SAR1, SSK, Sam-S, VhaSFD, Sec61α, Nito, snRNP, Rpn11, Rpn12, Ebony, Surf4, Prosα1, Prosα6, Uba1, Chc, Shi, ATPsyn-β, Cas, Prosβ5, RpL6, Main, unc-104, DSP1, Fkh, Hel25E, Gcm, spt16, NCM, ROP, RPB7, DRE4, RP11140, snf7, Sac1, actin, and iap. When the prefix ds is used in front of a lethal gene as is well understood in the art, it refers to a dsRNA molecule or molecules that target said gene for knock down or knock out.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

As used herein, the term “subject” refers to a target of administration. The subject of the herein disclosed methods can be an insect. The subject of the herein disclosed methods can include an insect of the order beetles (Coleoptera), of the family Scarabaeidae, or the subfamily Rutelinae. The subject of the herein disclosed methods can include an insect of the genus Popillia. The subject of the herein disclosed methods can include an insect of the species Popillia japonica.

As used herein, “synergy,” “synergistically,” “synergism,” and “synergistic effect” can refer to any substantial enhancement, in a composition of at least two compounds, of a measurable effect. In particular, synergism is a well-understood feature in the art, where components of a composition potentiate each other's effect. Synergy is a specific feature of a combination of components, and is above any background level of enhancement that would be due solely to, e.g., additive effects of any random combination of ingredients.

The presently-disclosed subject matter is further illustrated by the following specific but non-limiting examples. The following examples may include compilations of data that are representative of data gathered at various times during the course of development and experimentation related to the present invention.

### EXAMPLES

### Example 1: Insect Collection and Rearing Method

The adult Japanese beetles used in this study were field-collected by hand from the University of Kentucky's organic south farm in Lexington, Kentucky. The beetles were maintained in BugDorm-1 screen cages inside of an incubator chamber with conditions of 23±2° C., 80-90% relative humidity, and a photoperiod of 14 h light/10 h dark. The beetles were provided with fresh linden leaves (Tilia cordata Mill.) for 24 hrs after collection, then starved overnight prior to conducting assays the following day.

### Example 2: Total RNA Extraction and RT-PCR Method

Total RNA was isolated from Japanese beetle using Trizol reagent according to the manufacturer's instruction. Two micrograms of RNA was used for cDNA synthesis. The synthesized cDNA was added to a PCR 2×master mix and thermal cycling was performed using gene specific primers flanked with Hind III and Xba I in forward and reverse primers specific to actin gene to amplify a 300 bp fragment. Similarly, a 250 bp fragment of a gene coding for green fluorescent protein (GFP) was also amplified for use as control. A The thermal cycling conditions: 94° C. for 4 min, followed by 35 cycles of 94° C. for 30 s, 58° C. for 30 s and 72° C. for 45 s, finishing with an extension step at 72° C. for 10 min. The amplified PCR product was purified using the PCR purification kit (Qiagen Inc., Valencia, CA USA).

### Example 3: Construction of Plasmids for Expression of dsActin and dsGFP

For the construction of recombinant plasmid vector for expression of dsRNA targeting actin and GFP (control) the purified PCR products were digested with Hind III and Xba I restriction enzymes, and inserted into L4440 vector. The L4440 vector has two T7 RNA polymerase promoters at both ends of the multiple cloning sites in flanking orientation. The recombinant plasmids were transformed into competent cells of E. coli DH10 B cells. The positive clones were confirmed by colony PCR and plasmid DNA was isolated using plasmid mini preparation kit (Qiagen) according to manufacturer's instructions.

### Example 4: Preparation of Inactivated Bacteria

In vitro transcription is an expensive procedure and is not economical to synthesis large quantities of dsRNA that would be required for experiments involving insect bioassay. Hence, synthesizing dsRNA in RNase III deficient bacterial cells was attempted. HT115 (DE3) is a strain of E. coli which has been genetically engineered to enable synthesis of dsRNA. HT115 (DE3), a genetically engineered strain of bacteria which is deficient for RNase III. Competent cells of HT115 (DE3) cells were prepared and the plasmids L4440-Actin and L4440-GFP were transformed in the competent cells. dsRNA synthesis in bacteria and isolation was done according to the method of Timmons et al., (2001). Transformed HT115 (DE3) cells were inoculated in Luria-Bertani (LB) medium and cultured overnight at 37° C. The culture was diluted 1:100 fold and allowed to grow to OD600=0.4−0.6. Isopropyl β-D-1-thiogalactopyranoside (IPTG) was added to a final concentration of 0.4 mM and the culture was incubated with shaking for 4 h at 37° C. After 4 h, the cells were harvested by centrifugation and total nucleic acid were extracted. The expressed dsRNA was confirmed by 1% agarose gel electrophoresis.

After induced overexpression of dsRNA, the cultured broth was centrifuged at 2500 rpm for 10 mins and supernatant was removed. The pellet was washed with 1×PBS by centrifugation. Then dry the pellet and dissolved in 2 ml of sterile water. The bacteria were heat killed at 80° C. for 30 min. Prior to feeding, the bacterial viability was assessed by plating 100 μl of the heat killed bacterial sample on LB agar plates containing antibiotics both ampicillin and tetracyclin.

To assess the insecticidal activity of the recombinant bacteria expressing dsRNA, 2 ml of bacterial suspensions were coated on fresh linden (Tilia cordata Mill.) and rose plant (Rosa sp.) leaves and fed to beetle (N=10). Bacterially expressed dsGFP used as a negative control. The insect feeding bioassay was performed under controlled conditions at 25±2° C., 50-60% relative humidity, and a photoperiod of 14 h light/10 h dark. The feed was refreshed and mortality rates were observed daily for 7 days. For knockdown assay, adult beetles were collected after three days of feeding for qPCR analysis.

### Example 5: Quantitative Real-Time PCR (RT-qPCR)

Total RNA was extracted using Trizol reagent. Two micrograms of total RNA was used for first-strand cDNA synthesis using M-MLV reverse transcriptase (Invitrogen, USA). This cDNA was used as a template for RT-qPCR. Each RT-qPCR reaction contained 5 μl of iTaq™ universal SYBR Green Master Mix, 2 μl of 1:3 ratio diluted cDNA and 0.5 μl each of 10 μM forward (GACCAACAATGGATGGGAAGA—SEQ ID NO: 1) and reverse (CGTAGACAATGGATCCGGTATG—SEQ ID NO: 2) gene-specific primers. Amplification conditions are as follows: 95° C. for 20 s, followed by 40 cycles of 95° C. for 5 s, 60° C. for 20 s. At the end of each RT-qPCR reaction, a melting curve was generated to confirm a single peak and rule out the possibility of primer-dimer and non-specific product formation. The RPL32 was used as a reference gene and 2^{-ΔΔCt} method was used to calculate the relative expression level of the target gene in the samples as compared to controls. Statistical significance was determined by one-way ANOVA with Turkey post hoc analysis.

### Example 6: Bacterially-Expressed dsRNA

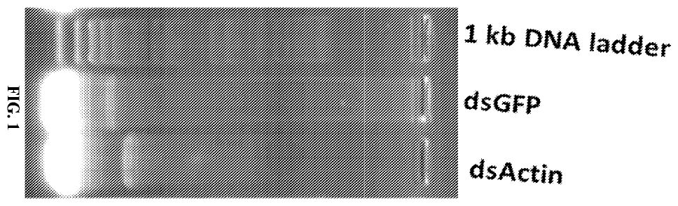

The bacterially expressed dsRNA was synthesized using L4440 plasmid inserted target gene of interest. The recombinant plasmid was transformed into HT115 (DE3) RNase III deficient E. coli cells. The transformed bacteria was induced by IPTG to overexpress dsRNA, which was confirmed by 1% agarose gel electrophoresis (FIG. 1).

### Example 7: Activity Against Japanese Beetle

To test efficacy of insecticidal activity of a bacterially expressed dsRNA against the Japanese beetle, linden (Tilia cordata Mill.) and rose plant (Rosa sp.) leaves were coated with inactivated bacteria containing E. coli expressed dsRNA targeting dsActin or dsGFP (Control) then fed to the adult beetles. Single stems of either linden or rose that were of approximately the same size were placed in a small flask containing 50 ml of water sealed with parafilm around the stem. Total 2 ml of inactivated bacteria was evenly coated on each leaf of the stem (9 leaves per linden stem, 20 leaves per rose stem). After the leaves had dried, 10 adult Japanese beetles were released and allowed to freely feed on the treated plant material.

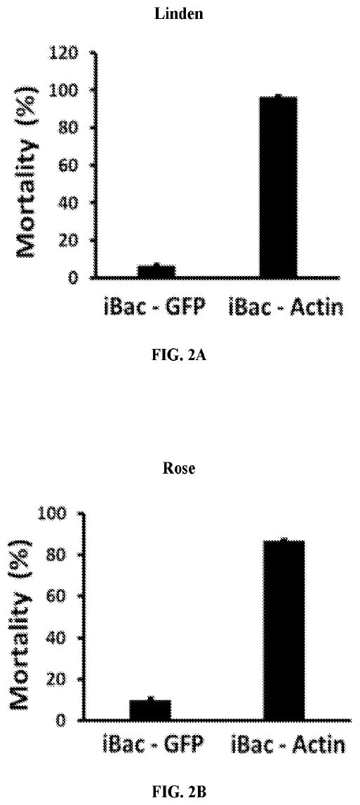

Mortality was scored daily, and after six days of feeding, beetles in the control group had consumed all of the leaf material with 10% mortality observed. Suppressed feeding activity and high mortality (100% in linden and 90% in rose plant, FIGS. 2A and 2B) was observed in beetles that were fed leaves treated with inactivated bacteria expressing dsActin. The results indicated that applying inactivated bacteria expressing dsActin could protect linden and rose plants from Japanese beetles.

### Example 8: Knockdown Efficiency of Ingesting Inactivated Bacteria Expressing dsActin

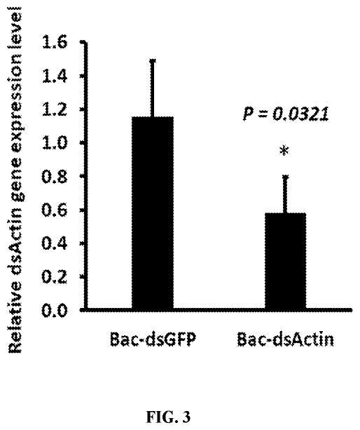

To confirm that bacterially expressed dsRNA was introduced into the beetles through feeding, mRNA expression levels were examined to verify knockdown of Actin in the insects. After three days of feeding, the beetles were collected and transcript levels were measured by RT-qPCR. Here, RPL32 was used as a reference gene to quantify mRNA levels. The results showed that inactivated bacteria expressing dsActin that was ingested by beetles showed significant knockdown efficiency (FIG. 3). These results demonstrate that bacteria were indeed ingested by the beetles, which resulted in knockdown of the target gene expression.

RNAi induction by dsRNA produced in bacteria was first demonstrated in Caenorhabditis elegans (Timmons et al. 2001). Application of this technology to control insect pests has been tried in a Leptinotarsa decemlineata (Zhu et al. 2011), Asian longhorn beetle (Dhandapani et al. 2020) and Spodoptera exigua (Tian et al. 2009). Similarly, the current study showed that E. coli expressing dsActin showed significant mortality and knockdown in Japanese beetle. This is a viable method to control invasive pest, the Japanese beetle.

### Example 9: Knockdown Efficiency of Ingesting Inactivated Bacteria Expressing dsRNA to Other Lethal Genes of Japanese Beetle

dsRNA directed towards other lethal genes of the Japanese Beetle are expected to work similar to dsActin. The Lethal genes of the Japanese Beetle are listed in Table 1.

TABLE 1
Lethal Genes

| | | | Size | Accession |
|---|---|---|---|---|
| Gene | Forward | Reverse | bp | Number |
| cactus | GGCCCTGTGACTTGGATCAAT | TTGGAACAGAAAACATGCGA | 404 | XM_018716333.1 |
| | (SEQ ID NO: 3) | (SEQ ID NO: 4) | | |
| asnap | GGTTATCAAAGGGCAGCCAATC | GGCTGAGGAAGCCACTTCTTGG | 443 | XM_018722883.2 |
| | (SEQ ID NO: 5) | (SEQ ID NO: 6) | | |
| sinra | GGTGGTGGTCTCCCTAAACCTG | GGTTTCTGTTCCTTTCCGGATG | 505 | XM_018709685.1 |
| | (SEQ ID NO: 7) | (SEQ ID NO: 8) | | |
| hsp | GGATAGGAGACGCAGCCAAGAA | GGTCAAAGTCCTCTCCTCCC | 550 | XM_018710750.1 |
| | (SEQ ID NO: 9) | (SEQ ID NO: 10) | | |
| gw | GGAGAATGCTCGTCGATATGGG | GGCCAACGGTTGATTGAGAAT | 482 | XM_023456527.1 |
| | (SEQ ID NO: 11) | (SEQ ID NO: 12) | | |
| srp | GGTTTGTCGGTTTGCAAGGTT | GGATCACGGAACCGACGTCTAC | 423 | XM_018721226.1 |
| | (SEQ ID NO: 13) | (SEQ ID NO: 14) | | |
| rop | GGTCCTCGACCCTAAAATCACG | GGCTGGGCATTTGTAACCTCGT | 500 | XM_018714711.1 |
| | (SEQ ID NO: 15) | (SEQ ID NO: 16) | | |
| ppla | GGAGAACGACAGGGGAGTGAG | GGTAACTTTTTCCGCATCAGGG | 435 | XM_018710110.1 |
| | (SEQ ID NO: 17) | (SEQ ID NO: 18) | | |
| rpn7 | GGAGACAAAGAGAACGCCGTC | GGTAACGAAAGGTTCCGAGTGG | 426 | XM_018724821.1 |
| | (SEQ ID NO: 19) | (SEQ ID NO: 20) | | |
| rpt3 | GGATGAACAGCGCAACTTGAA | GGACGGCCTTAGCAAGCATAG | 471 | XM_018705385.1 |
| | (SEQ ID NO: 21) | (SEQ ID NO: 22) | | |
| Mesh | GAGAAGTTAGGGCAACGCAG | CCAGTTTCCAAAAAGTCCCA | 341 | XM_018707459.2 |
| | (SEQ ID NO: 23) | (SEQ ID NO: 24) | | |
| HEL25E | TTTGTGCCAAGCAAAGTCTG | CCAGCATTTTGTCACATTCG | 353 | XM_018720653.1 |
| | (SEQ ID NO: 25) | (SEQ ID NO: 26) | | |
| Sec23 | GGCTGTAATAATGGCGAGGA | TCTCCCCATGGAAGATCAAG | 422 | XM_018715734.2 |
| | (SEQ ID NO: 27) | (SEQ ID NO: 28) | | |
| SAR1 | GGGTTGGATAATGCAGGAAA | GCCCGTACAAGGCATAGAAA | 421 | XM_018718347.1 |
| | (SEQ ID NO: 29) | (SEQ ID NO: 30) | | |
| SSK | GGGAGGAACATGGAATTTGA | CGGAGTCAATGAGATACGCC | 326 | XM_018724815.1 |
| | (SEQ ID NO: 31) | (SEQ ID NO: 32) | | |
| Sam-S | CACAGAGCGATGCAGGTTTA | GTTTAGGCGTAGCACCTTCG | 425 | XM_018717099.1 |
| | (SEQ ID NO: 33) | (SEQ ID NO: 34) | | |
| VhaSFD | GCCGACATCTTAAGCGACTC | AGTACGAGGGGATCCTTGCT | 410 | XM_018721020.1 |
| | (SEQ ID NO: 35) | (SEQ ID NO: 36) | | |
| Sec61α | GATCGAGCTCTTTTCAACGG | ACGATGGCAAAAACGAAAAC | 443 | XM_018707923.1 |
| | (SEQ ID NO: 37) | (SEQ ID NO: 38) | | |
| Nito | TTCCGTAATCCAGAGGATGC | GGGCGTGGAACTGTATGTCT | 377 | XM_018715236.1 |
| | (SEQ ID NO: 39) | (SEQ ID NO: 40) | | |
| snRNP | AAAAATCGGGGTTATTTGGG | ATGCATGTCACGTTCGTGTT | 375 | XM_018721256.1 |
| | (SEQ ID NO: 41) | (SEQ ID NO: 42) | | |
| Rpn11 | GGTTTTGGGTGTTGGTTGTC | CTCCTCTTCTTCCAGGGCTT | 441 | XM_018719490.1 |
| | (SEQ ID NO: 43) | (SEQ ID NO: 44) | | |
| Rpn12 | GTGGAGCATTGCAGCAAATA | TGGCAGCCTCTTCACTAGGT | 440 | XM_018724257.1 |
| | (SEQ ID NO: 45) | (SEQ ID NO: 46) | | |
| Ebony | AAAATTAAAACGCAACCCCC | TTGGGAGAGAGGGATGAATG | 413 | XM_018713129 |
| | (SEQ ID NO: 47) | (SEQ ID NO: 48) | | |
| Surf4 | TCTTGGAAGATGGGTTACGC | AATTTTTGGGCTTGTTGTCG | 357 | XM_018716988 |
| | (SEQ ID NO: 49) | (SEQ ID NO: 50) | | |
| Prosα1 | CGGATTCTATTGGATGCGTT | TCATGCTGCAACTCTTGCTT | 358 | XM_018718366.1 |
| | (SEQ ID NO: 51) | (SEQ ID NO: 52) | | |
| Prosα6 | GGGCTTGAAGAGCAACAGTC | TGGACATAGCTTTGCAGTCG | 374 | XM_018714266.1 |
| | (SEQ ID NO: 53) | (SEQ ID NO: 54) | | |
| Uba1 | AGCAGCAGATCGGCATAAGT | ACTTCGGACATGGTGAGACC | 408 | XM_018717783.1 |
| | (SEQ ID NO: 55) | (SEQ ID NO: 56) | | |
| Chc | CGACATCGCTAACATCGCTA | CGGACTCGATGTAGCTCTCC | 356 | XM_018721034.1 |
| | (SEQ ID NO: 57) | (SEQ ID NO: 58) | | |
| Shi | ACATGTTGCCTTTAGACGGG | CTGGCGTAAACGTGTGCTAA | 427 | XM_018714700.1 |
| | (SEQ ID NO: 59) | (SEQ ID NO: 60) | | |
| ATPsyn-β | AGGATTGGTACGTGGACAGC | CAAGGAGATGACACCGGACT | 424 | XM_018706757.1 |
| | (SEQ ID NO: 61) | (SEQ ID NO: 62) | | |
| Cas | TGCTCAACTGCAACCGATAG | AACTGCATGTAAGGTTCGGG | 429 | XM_018707893.1 |
| | (SEQ ID NO: 63) | (SEQ ID NO: 64) | | |
| Prosβ5 | ACGATGTGAGCAAGTCGTGT | GGCAGCTGCTACTGAAATCC | 377 | XM_018713596.1 |
| | (SEQ ID NO: 65) | (SEQ ID NO: 66) | | |
| RpL6 | ACGGAGGGAAGAGATTCGTT | TTCCTCTGGTCGCTGACTTT | 433 | XM_018709657.1 |
| | (SEQ ID NO: 67) | (SEQ ID NO: 68) | | |
| Mam | ACCTGATAATGATTTCGCGG | GTTTGAGCGGCTGGACTTAG | 441 | XM_018717665.1 |
| | (SEQ ID NO: 69) | (SEQ ID NO: 70) | | |
| unc-104 | CAAAGTGTACGCCCCATCTT | CCTTGCTTTTCCAACAGCTC | 406 | XM_018711981.1 |
| | (SEQ ID NO: 71) | (SEQ ID NO: 72) | | |
| DSP1 | CCACCAGCGAGGATAGTGAT | TGCGGCTTCTGTTTTCTTTT | 323 | XM_018711607.1 |
| | (SEQ ID NO: 73) | (SEQ ID NO: 74) | | |
| Fkh | TCATTTTGGAGCCTTCATCC | CCTGCAGGTTCCTGCTTTAG | 449 | XM_018721646.1 |
| | (SEQ ID NO: 75) | (SEQ ID NO: 76) | | |
| He125E | AAGACCCGTGAAGCTGAAGA | TTGTGCCTCAATTCCCTTTC | 428 | XM_018708786.1 |
| | (SEQ ID NO: 77) | (SEQ ID NO: 78) | | |
| Gcm | CAAAGCGTCAATGTCGAGAA | TGGTGCCACTTCCTAGATCC | 417 | XM_018711698.1 |
| | (SEQ ID NO: 79) | (SEQ ID NO: 80) | | |
| sptl6 | AAGACCCGTGAAGCTGAAGA | TTGTGCCTCAATTCCCTTTC | 428 | XM_018708786.1 |
| | (SEQ ID NO: 81) | (SEQ ID NO: 82) | | |
| NCM | TTCTAGAACAGGCGGTGCTT | ATGGCCACCTCAACAGAATC | 504 | XM_018712415.1 |
| | (SEQ ID NO: 83) | (SEQ ID NO: 84) | | |
| ROP | TGCATGTACAAAGCAGCACA | GGGATCTAGCCTTTTCTGGG | 397 | XM_018714711.1 |
| | (SEQ ID NO: 85) | (SEQ ID NO: 86) | | |
| RPB7 | GGCCTAATACTGCCTGGTCA | ATGCCAGAAGCATCCACTCT | 302 | XM_018719020.2 |
| | (SEQ ID NO: 87) | (SEQ ID NO: 88) | | |
| DRE4 | TATACTCAACCCGAGGCGAC | GACGTGTAACGGAAGCCATT | 314 | XM_018708786.1 |
| | (SEQ ID NO: 89) | (SEQ ID NO: 90) | | |
| RP11140 | CTTCGGTTCAGAGAGTTGCC | CGGGTAGCGTCATTGTTTTT | 325 | XM_018712425.1 |
| | (SEQ ID NO: 91) | (SEQ ID NO: 92) | | |
| snf7 | GGGTTGCGCTTCAAGCCCTT | CCAGACCTTCCTGTTCCAGCTC | 342 | XM_018722997.1 |
| | (SEQ ID NO: 93) | (SEQ ID NO: 94) | | |
| Sac1 | GTAGCCGAATTGTACGGGAA | AATTGCGATGTGCCAACATA | 340 | XM_018707373.1 |
| | (SEQ ID NO: 95) | (SEQ ID NO: 96) | | |
| actin | GAAAGGGAAATTGTCCGTGA | AGAGGGAAGCCAAGATGGAT | 436 | XM_018721905.1 |
| | (SEQ ID NO: 97) | (SEQ ID NO: 98) | | |
| iap | TCCTCGCCGACAAAATAATC | CCTCGGAATGATCGTGTTCT | 386 | XM_018711271.2 |
| | (SEQ ID NO: 99) | (SEQ ID NO: 100) | | |

### Example 10: Mortality of Beetles Following Application of dsRNA to Plants

Okra Plants were planted, including 480 in each plot. Treatment Groups were separated by 10 Buffer Plants (unsprayed). Prior to spraying and starting the experiment, all Okra fruits were removed from every plant in the plot. All weeds are removed from each plot prior to starting and prior to any follow-up application. 1.8 L of each treatment was sprayed on 40 plants during each application event (45 ml/plant).

Application events occurred as follows:
Spray #1 (Start): Day 1
Spray #2: Day 8
Spray #3: Day 16
Spray #4: Day 22-24. Weather Permitting

Treatments were as follows:
Control (H₂O Sprayed): 10×5 Plants
dsGFP Bacteria (Weekly Application): 10×4 Plants
dsActing Bacteria (Single Application): 10×4 Plants
dsActin Bacteria (Weekly Application) 10×4 Plants
Carbaryl (Applied Day 1, Day 16): 10×4 Plants
   *30 ml of Carbaryl added to 1.8 L of H₂O
Btg (Applied 5 days before Day 1, Day 16): 10×4 Plants
   *2 oz of Btg powder added to 1.8 L of H2O
dsActin Bacteria+Nu-Film P (Weekly, Started Day 8): 10×1 Plants
   *2 mL of Nu-Film P added to 1.8 L of dsActin Bacteria

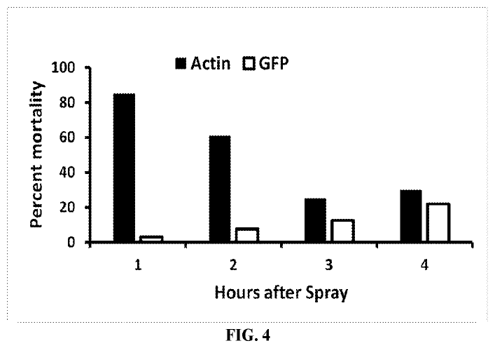

Visual observations did not show significant differences among treatments. Leaves from treated and control plots were collected and assayed them in the lab. As shown in FIG. 4, leaves collected from dsActin-treated plots caused >80% mortality at 24 hr after treatment and the mortality decreased over a two-week period to 30%. In contrast, the leaves from control dsActin-treated plots caused 2% mortality at 24 hr after treatment and the mortality increased to 20%. These data show that dsActin sprayed on plants kills beetles, without damaging the plant.

### Example 11: Mortality of Beetles Following Application of dsRNA to Plants

Stems of linden or okra were selected of equal size with five leaves (linden) or approximately 10 mm×10 mm leaf area (okra). Triton-X (diluted 1:1000) was lightly painted on each leaf surface and allowed to dry. 1 ml of Carbaryl, heat-killed bacteria expressing dsGFP or dsActin, or heat-killed bacteria expressing bacteria and adjuvants (“Adj”; Nu-Film P and Monterey LG 6286 Horticultural Oil) were applied to the front and back of leaves on each stem.

Stems were placed in a small flask of water, and the entire flask and opening were covered in parafilm to create a vase that beetles can climb on to access food. A total of 5 female beetles were released on each stem (total of 2 stems and 10 female beetles per treatment in each experimental setup). After 3 days when all leaves are consumed and insects fed untreated linden or okra while continuing to monitor. Carbaryl, Horticultural Oil and Nu-Film P spreader sticker were applied after being diluted and prepared according to the manufacturer's instructions.

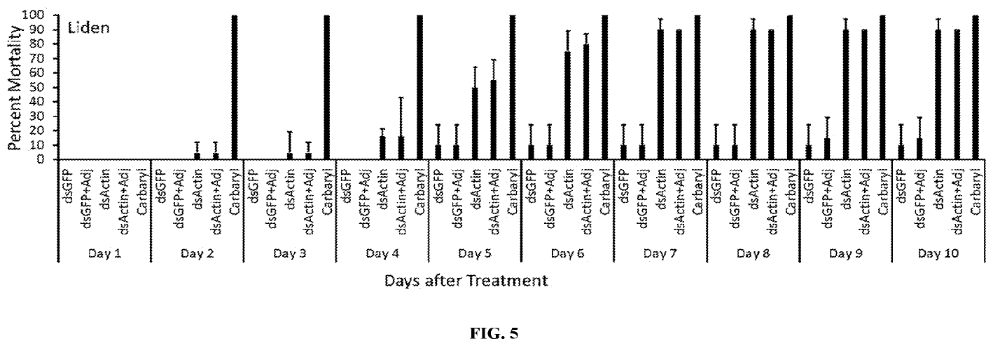

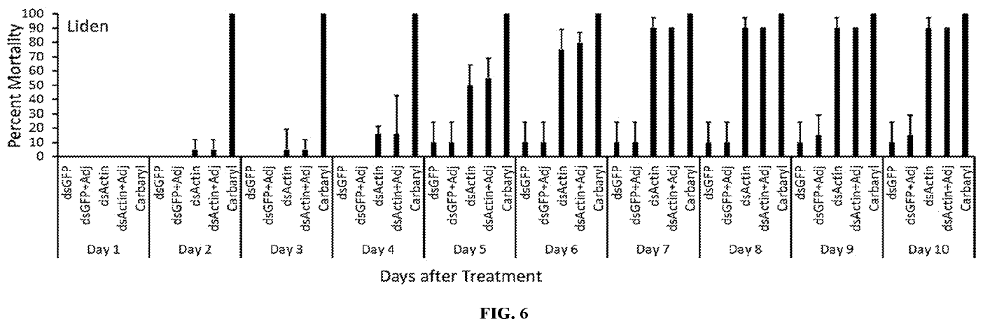

FIGS. 5 and 6 include data showing the Mortality of beetles fed on linden leaves treated with dsGFP (control), dsGFP plus adjuvant (ad) (Control), dsActin, dsActin plus adjuvant (Ad) and Carbaryl (positive control). The beetles alive and feeding were counted on each day after treatment and the percent mortality was calculated. Mean and standard deviation of two duplicate experiments are shown.

As expected, Carbayl, a chemical insecticide used to control beetles caused 100% mortality by the second day after treatment. The dsActin induced mortality of beetles began on day 5 and reached 100% by day 7 after treatment (FIGS. 5 and 6). The mortality trends are similar on linden and rose plant treatments.

### Example 12: Stability of dsRNA on Plants

Okra leaves of approximately equal size (˜15 cm length) were collected from either Bac-dsGFP or Bac-dsActin treated plots at given time points after application of treatment and stored at 4 C. All treated leaves also had both adjuvants (NuFilm P and Horticultural Oil).

Adult Japanese beetles were also collected from these plots to be used for the assay. Mortality was monitored daily for 10 days.

In addition, after 4 days of feeding a subset of beetles were sacrificed for knockdown study. The objective of this assay was to determine efficacy of the application at different time points to determine if the dsRNA enters the plant systemically after spraying and for how long the treatment is effective at killing or silencing target gene expression after initial spray.

The initial spray treatment was performed on Day 1 and assays were conducted at 24 hrs, 48 hrs, 72 hrs, 96 hrs and 1-week post-spray.

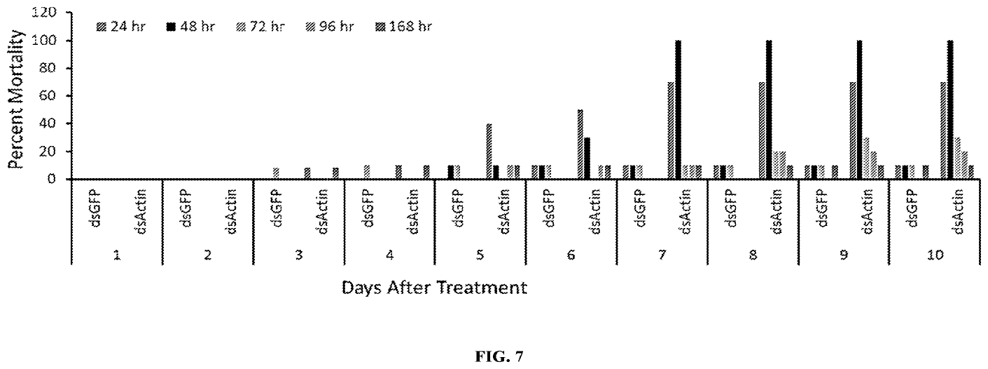

With reference to FIG. 7, Okra leaves collected at 24, 48, 72, 96 and 168 hr after spraying dsGFP or dsActin were fed to beetles, and mortality of beetles was recorded daily until 10 days after treatment. As shown, the dsActin spayed on okra plants in the field is active for 48 hr and induced mortality of beetles starting on day 5 after treatment.

### Example 13: Field Trial—Okra Plot

Okra (Jambalaya) was grown in greenhouse conditions then transplanted to a dedicated plot at the University of Kentucky's South Farm. The okra were planted 2 ft spacing between plants in 4 rows of 80 plants each. Prior to spraying and starting the experiment, all Okra fruits were removed from every plant in the plot. All weeds are removed from each plot prior to starting and prior to any follow-up application.

Two plots of 10 plants each were covered in the field plot using 8′ rebar stakes and 14′ wide, fine mesh horticultural netting (ProtekNet) to prevent insects from entering or escaping from the plot. These plots were treated every 4 days with either heat-killed bacteria expressing dsGFP or dsActin. The solutions also contained a 1:100 ratio by volume of two adjuvants: NuFilm P (spreader-sticker) and Monterrev Horticultural Oil (penetrator). Treatments were applied with a gas powered sprayer at a rate of approximately 50 ml per plant. 24 hrs after the first application, 50 female Japanese beetles were released into each of the enclosed plots on Day 1. An additional 50 female beetles were released into each of the enclosed plots on Day 15.

Five additional plots of 10 plants each were selected at random for the other treatments, including Carbaryl (positive control), heat-killed bacteria expressing dsGFP with adjuvants, heat-killed bacteria expressing dsActin, heat-killed bacteria expressing dsActin with adjuvants, and an untreated control plot. Starting on August 18^{th}, Carbaryl was replaced with Talstar P (Bifenthrin) as a positive control due to lack of efficacy in Carbaryl-treated plots. Foliage damage and beetle densities were estimated by two observers on each plot weekly.

Leaves and Japanese beetles were collected 24 hr, 48 hr, 72 hr, 96 hr, and 1 week after spraying for dsRNA stability analysis and knockdown studies, respectively. These samples were stored in Trizol in −80 C conditions for storage prior to analysis.

When Japanese beetle populations are no longer detectable in the field, leaf material from each plot is analyzed to quantity damage. Fruit yield is also compared between the treatment groups.

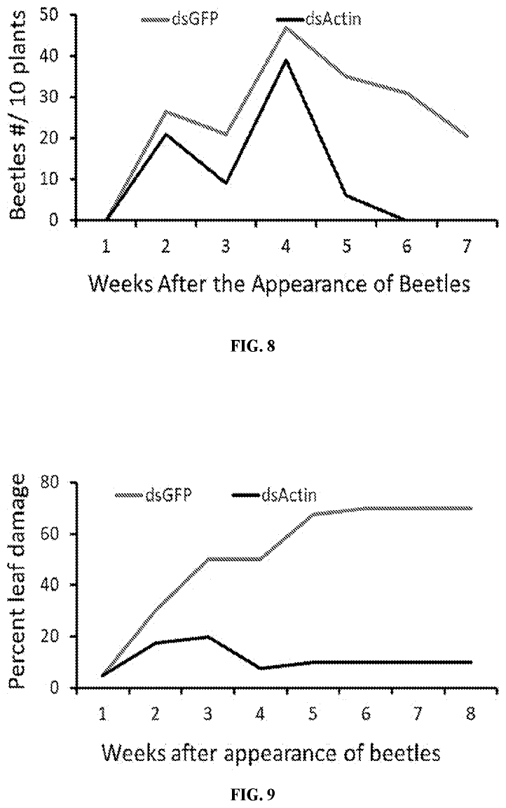

The number of beetles detected is presented in FIG. 8. The number of beetles was detected weekly for seven weeks after spraying dsGFP (control) or dsActin on Okra plants. The number of beetle counts on 10 plants per treatment are shown. As depicted, the appearance of beetles was consistently lower on the plants treated with dsActin as compared to control.

Leaf damage by beetles is shown in FIG. 9. The leaf damage by beetles was recorded weekly for seven weeks after spraying dsGFP (control) or dsActin on Okra plants. The percent leaf damage estimates on 10 plants per treatment are shown. As depicted, the percentage of leaf damage approached 80% in the control, while the dsActin plants displayed minimal leaf damage.

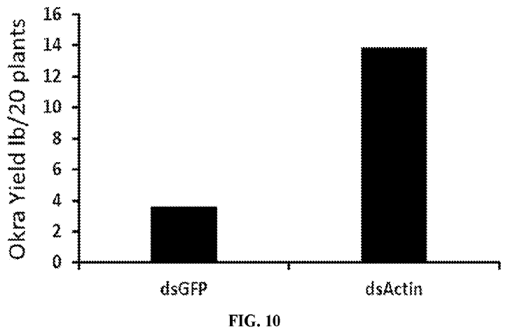

Okra yield quantification is presented in FIG. 10. Okra yield was quantified at the end of the season from 10 plants from dsGFP (control) and dsActin treatments. As shown, the okra yield in the dsActin plans was much higher as compared to control.

### Example 14: Field Trial—Rose Study

Eight knockout roses in 3-gallon containers of approximately equal size and foliage density were selected for the experiment. The roses were set up on private farmland property on flat terrain. Each container was staked into the ground using a rebar spike to prevent the plant from moving during windy conditions. Before starting the experiment, all but 3 flowers were pruned from each of the plants to mitigate differences in floral attractiveness to the insects.

The rose containers were set up in two rows of four plants each. The rows were 5 m apart from one another, and each container was spaced 2 m from one another in each row. A single TRÉCÉ JAPANESE BEETLE (JB) FLORAL LURE (Great Lakes IPM) was placed on each container to both attract Japanese beetles and encourage them to remain on the host plant. Each plant was sprayed with 150 ml of heat-killed E. coli bacteria expressing dsRNA targeting either GFP (control) or Actin every 4 days starting on Day 1. The solutions also contained a 1:100 ratio by volume of two adjuvants: NuFilm P (spreader-sticker) and Monterrey Horticultural Oil (penetrator).

Beetle densities and foliage damage estimates were documented by two observers once weekly. Leaves were collected 24 hr, 48 hr, 72 hr, 96 hr, and 1 week after spraying for dsRNA stability analysis. These samples were stored in Trizol in −80 C conditions to be analyzed at a later date.

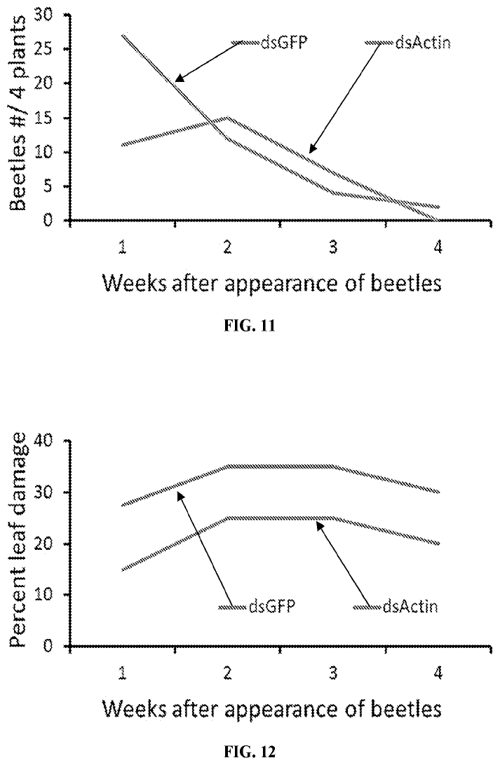

The number of beetles detected on the plans is depicted in FIG. 11. The number of beetles was detected weekly for seven weeks after spraying dsGFP (control) or dsActin on Okra plants. The number of beetle counts on 10 plants per treatment are shown. At week 1 there was a lower beetle count on dsActin plants, as compared to control, and at subsequent weeks, the numbers became similar, but both declined week-over-week.

With reference to FIG. 12, leaf damage by beetles was recorded weekly for seven weeks after spraying dsGFP (control) or dsActin on Okra plants. The percent leaf damage estimates on 10 plants per treatment are shown. As depicted, the leaf damage was consistently lower in the dsActin leaves as compared to control.

### Example 15: Stability of dsRNA in Japanese Beetle Lumen Content

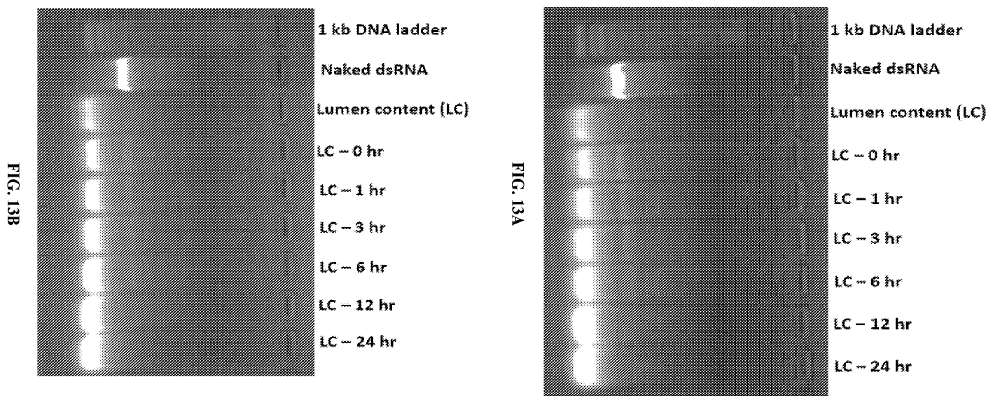

One microgram of dsRNA was exposed to 10 g of midgut lumen contents and incubated 0, 1, 3, 6, 12 and 24 hours in room temperature. The sample mixtures were collected and analyzed in 1% agarose gel electrophoresis. As illustrated by the data depicted in FIG. 13A-13C, the dsRNA is stable in Japanese beetle lumen content.

### Example 16: Identification of RNAi Target Genes in Japanese Beetle

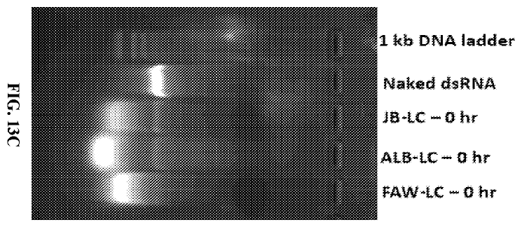

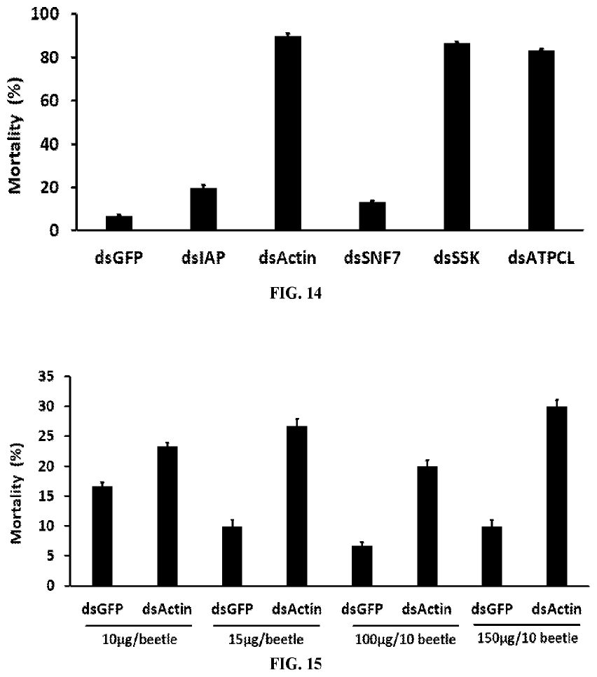

Ten micrograms of dsRNA targeting each gene was injected into each adult beetle. Mortality was scored on the 10^{th} day after injection of dsRNA. dsGFP was used as a control. With reference to FIG. 14, RNAi target genes were identified in Japanese beetle. The mortality rate for the three genes that showed the greatest efficiency was used in the screening assay.

### Example 17: Feeding Naked dsRNA by Linden Leaf in Japanese Beetle

A total of 10 μg/beetle, 15 g/beetle, 100 μg/10 beetle and 150 μg/10 beetle of dsGFP and dsActin were fed to adult beetles for 3 days. dsRNA was spread on 1 cm diameter leaf discs for individual beetles and 4 cm×4 cm leaf squares in group assays. The mortality was observed up to 10 days after feeding and percent mortality was recorded. With reference to FIG. 15, there was no significant mortality was observed in this assay (N=3).

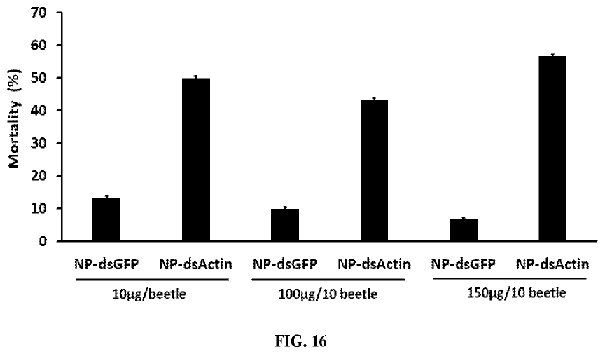

Example 18: Nanoparticle mediated RNAi in Japanese beetle by leaf feeding bioassay. PLL:EGCG:dsRNA nanoparticles were added to a 4 cm×4 cm square of linden leaf and adult beetles were allowed to feed (total 10 μg/beetle, 100 μg/10 beetle & 150 μg/10 beetle of dsGFP and dsActin) for 3 days. With reference to FIG. 16, the mortality was observed up to 10 days post feeding and percent mortality was recorded. (N=3).

### Example 19: RNAi Induced by Heat-Killed Bacteria Expressing dsActin in Japanese Beetle

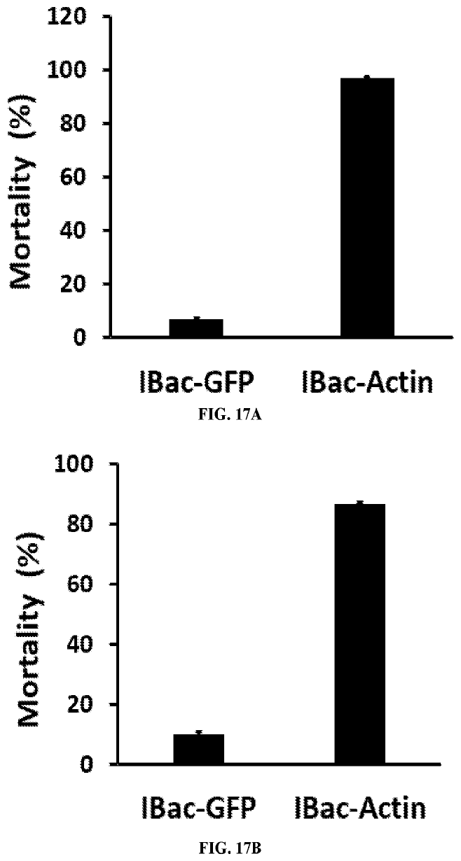

Heat inactivated bacteria expressing dsGFP or dsActin were sprayed on (FIG. 17A) linden leaves and (FIG. 17B) Rose, and the mortality was recorded on 6^{th} day post-feeding and percent mortality was recorded. (Mean S.D) (N=30).

### Example 20: Feeding of dsRNA after Knockdown of dsRNase's in Japanese Beetle

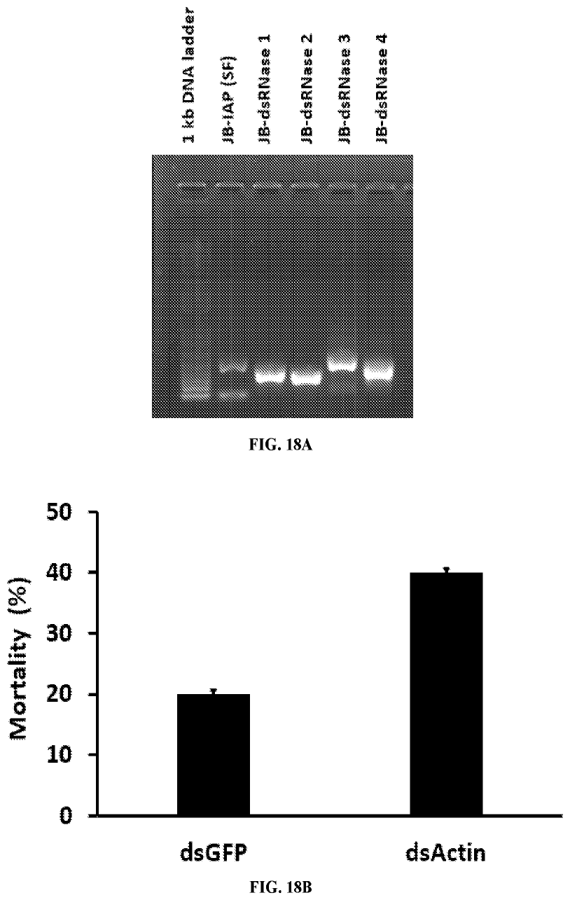

A total of 40 ug of mixed dsRNase's (1, 2, 3 & 4) were injected into each adult Japanese beetle. After three days, fifteen micrograms of dsGFP and dsActin were fed to the beetles for up to three days. The mortality was scored up to 10 days post-feeding. With reference to FIGS. 18A and 18B, there was no significant mortality was observed in this assay.

### Example 21: Feeding Bioassay with Japanese Beetle Exposed to Bacillus thuringiensis Toxin (Bt) and Bt-dsRNA

A total of 150 μg/10 beetle of Bt-dsGFP and Bt-dsActin was added to a linden leaf and beetles were allowed to feed up to three days. With reference to FIG. 19, after three days mortality was scored up to 10 days post-feeding.

### Example 22: Feeding Bioassay with Japanese Beetle Exposed to Bt and Bt/NP-dsRNA

A total of 150 μg/10 beetle of Bt/NP-dsGFP and Bt/NP-dsActin was added to a linden leaf and beetles were allowed to feed up to three days. With reference to FIG. 20. After three days mortality was scored up to 10 days post-feeding.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Tian, H., Peng, H., Yao, Q., Chen, H., Xie, Q., Tang, B., & Zhang, W. (2009). Developmental control of a lepidopteran pest Spodoptera exigua by ingestion of bacteria expressing dsRNA of a non-midgut gene. PLoS one, 4(7), e6225.
2. Timmons, L., Court, D. L., & Fire, A. (2001). Ingestion of bacterially expressed dsRNAs can produce specific and potent genetic interference in Caenorhabditis elegans. Gene, 263(1-2), 103-112.
3. Zhu, F., Xu, J., Palli. R., Ferguson, J., & Palli. S. R. (2011). Ingested RNA interference for managing the populations of the Colorado potato beetle, Leptinotarsa decemlineata. Pest management science, 67(2), 175-182.
4. Dhandapani, R. K., Gurusamy. D., Duan, J. J., & Palli, S. R. (2020). RNAi for management of Asian long-homed beetle, Anoplophora glabripennis: identification of target genes. Journal of Pest Science. 93(2), 823-832.
5. Whitten, M. M. A. (2019) Novel RNAi delivery systems in the control of medical and veterinary pests. Current Opinion in Insect Science. 34, 1-6, doi.org/10.1016/j.cois.2019.02.001.
6. Basnet S, Kamble S T. (2018) RNA Interference of the Muscle Actin Gene in Bed Bugs: Exploring Injection Versus Topical Application for dsRNA Delivery. J Insect Sci. 18(3):5. doi: 10.1093/jisesa/iey045. PMID: 29788394: PMCID: PMC5961172.
7. Joga, M. R.: Mogilicherla, K.; Smagghe, G.; & Roy, A. (2021) RNA Interference-Based Forest Protection Products (FPPs) Against Wood-Boring Coleopterans: Hope or Hype? Front. Plant Sci., 12, doi.org/10.3389/fpls.2021.733608.
8. Xu, L., Xu, S., Sun, L. et al. (2021) Synergistic action of the gut microbiota in environmental RNA interference in a leaf beetle. Microbiome 9, 98. doi.org/10.1186/s40168-021-01066-1.
9. U.S. Pat. No. 11,571,394 for modified-RNA nanoparticles for induction of RNA interference.

It will be understood that various details of the presently disclosed subject matter can be changed without departing from the scope of the subject matter disclosed herein. Furthermore, the foregoing description is for the purpose of illustration only, and not for the purpose of limitation.

1. A composition, comprising: inactivated bacteria containing expressed dsRNA targeting a lethal gene of an insect of the genus Popillia, or a nanoparticle containing dsRNA targeting a legal gene of an insect of the genus Popollia.

2. The composition of claim 1, and further comprising an adjuvant.

3-4. (canceled)

5. The composition of claim 1, wherein the insect is of the species Popillia japonica.

6. The composition of claim 1, wherein the legal gene is selected from the group consisting of insect lethal genes such as Cactus, asnap, sinra, hsp, gw, srp, rop, pp1a, rpn7, rpt3, Mesh, HEL25E, Sec23, SAR1, SSK, Sam-S, VhaSFD, Sec61α, Nito, snRNP, Rpn11, Rpn12, Ebony, Surf4, Prosα1, Prosα6, Uba1, Chc, Shi, ATPsyn-β, Cas, Prosβ5, RpL6, Mam, unc-104, DSP1, Fkh, Hel25E, Gem, spt16, NCM, ROP, RPB7, DRE4, RP11140, snf7, Sac1, actin, Iap, and combinations thereof.

7. The composition of claim 1, and further comprising Bacillus thuringiensis toxin (Bt).

8. A method of inducing RNAi in an insect of the genus Popillia, comprising: administering to the insect a composition comprising inactivated bacteria containing expressed dsRNA targeting a lethal gene of the insect of the genus Popillia, or a nanoparticle containing dsRNA targeting a legal gene of an insect of the genus Popollia.

9. The method of claim 8, wherein the administration is oral.

10. The method of claim 9, and further comprising depositing the composition on a surface in an environment of the insect.

11. The method of claim 10, wherein the surface is a plant.

12. The method of claim 8, and further comprising re-depositing the composition on the surface every 1, 2, 3, 4, 5, 6, or 7 days.

13-14. (canceled)

15. The method of claim 8, wherein the insect is of the species Popillia japonica.

16. The method of claim 8, wherein the legal gene is selected from the group consisting of Cactus, asnap, sinra, hsp, gw, srp, rop, pp1a, rpn7, rpt3, Mesh, HEL25E, Sec23, SAR1, SSK, Sam-S, VhaSFD, Sec61α, Nito, snRNP, Rpn11, Rpn12, Ebony, Surf4, Prosα1, Prosα6, Uba1, Chc, Shi, ATPsyn-β, Cas, Prosβ5, RpL6, Mam, unc-104, DSP1, Fkh, Hel25E, Gem, spt16, NCM, ROP, RPB7, DRE4, RP11140, snf7, Sac1, actin, Iap, and combinations thereof.

17. The method of claim 8, wherein the composition further comprises Bacillus thuringiensis toxin (Bt).

18. A method of protecting a plant from an insect of the genus Popillia, comprising: applying to the plant a composition comprising inactivated bacteria containing expressed dsRNA targeting a lethal gene of the insect of the genus Popillia, or a nanoparticle containing dsRNA targeting a legal gene of an insect of the genus Popollia.

19. The method of claim 18, and further comprising re-depositing the composition on the surface every 1, 2, 3, 4, 5, 6, or 7 days.

20-21. (canceled)

22. The method of claim 18, wherein the insect is of the species Popillia japonica.

23. The method of claim 18, wherein the legal gene is selected from the group consisting of Cactus, asnap, sinra, hsp, gw, srp, rop, pp1a, rpn7, rpt3, Mesh, HEL25E, Sec23, SAR1, SSK, Sam-S, VhaSFD, Sec61α, Nito, snRNP, Rpn11, Rpn12, Ebony, Surf4, Prosα1, Prosα6, Uba1, Chc, Shi, ATPsyn-β, Cas, Prosβ5, RpL6, Mam, unc-104, DSP1, Fkh, Hel25E, Gem, spt16, NCM, ROP, RPB7, DRE4, RP11140, snf7, Sac1, actin, Iap, and combinations thereof.

24. The method of claim 18, wherein the composition further comprises Bacillus thuringiensis toxin (Bt).

25. The method of claim 24, and further comprising detection of synergistic protection of the plant, as compared to use of dsRNA or Bt alone.

26-50. (canceled)


SEQUENCE LISTING (100 sequences)

SEQ ID NO: 1 - DNA, 21 bp, synthetic construct
  source 1..21: mol_type = other DNA
gaccaacaatggatgggaaga

SEQ ID NO: 2 - DNA, 22 bp, synthetic construct
  source 1..22: mol_type = other DNA
cgtagacaatggatccggtatg

SEQ ID NO: 3 - DNA, 21 bp, synthetic construct
  source 1..21: mol_type = other DNA
ggccctgtgacttggatcaat

SEQ ID NO: 4 - DNA, 20 bp, synthetic construct
  source 1..20: mol_type = other DNA
ttggaacagaaaacatgcga

SEQ ID NO: 5 - DNA, 22 bp, synthetic construct
  source 1..22: mol_type = other DNA
ggttatcaaagggcagccaatc

SEQ ID NO: 6 - DNA, 22 bp, synthetic construct
  source 1..22: mol_type = other DNA
ggctgaggaagccacttcttgg

SEQ ID NO: 7 - DNA, 22 bp, synthetic construct
  source 1..22: mol_type = other DNA
ggtggtggtctccctaaacctg

SEQ ID NO: 8 - DNA, 22 bp, synthetic construct
  source 1..22: mol_type = other DNA
ggtttctgttcctttccggatg

SEQ ID NO: 9 - DNA, 22 bp, synthetic construct
  source 1..22: mol_type = other DNA
ggataggagacgcagccaagaa

SEQ ID NO: 10 - DNA, 20 bp, synthetic construct
  source 1..20: mol_type = other DNA
ggtcaaagtcctctcctccc

SEQ ID NO: 11 - DNA, 22 bp, synthetic construct
  source 1..22: mol_type = other DNA
ggagaatgctcgtcgatatggg

SEQ ID NO: 12 - DNA, 21 bp, synthetic construct
  source 1..21: mol_type = other DNA
ggccaacggttgattgagaat

SEQ ID NO: 13 - DNA, 21 bp, synthetic construct
  source 1..21: mol_type = other DNA
ggtttgtcggtttgcaaggtt

SEQ ID NO: 14 - DNA, 22 bp, synthetic construct
  source 1..22: mol_type = other DNA
ggatcacggaaccgacgtctac

SEQ ID NO: 15 - DNA, 22 bp, synthetic construct
  source 1..22: mol_type = other DNA
ggtcctcgaccctaaaatcacg

SEQ ID NO: 16 - DNA, 22 bp, synthetic construct
  source 1..22: mol_type = other DNA
ggctgggcatttgtaacctcgt

SEQ ID NO: 17 - DNA, 21 bp, synthetic construct
  source 1..21: mol_type = other DNA
ggagaacgacaggggagtgag

SEQ ID NO: 18 - DNA, 22 bp, synthetic construct
  source 1..22: mol_type = other DNA
ggtaactttttccgcatcaggg

SEQ ID NO: 19 - DNA, 21 bp, synthetic construct
  source 1..21: mol_type = other DNA
ggagacaaagagaacgccgtc

SEQ ID NO: 20 - DNA, 22 bp, synthetic construct
  source 1..22: mol_type = other DNA
ggtaacgaaaggttccgagtgg

SEQ ID NO: 21 - DNA, 21 bp, synthetic construct
  source 1..21: mol_type = other DNA
ggatgaacagcgcaacttgaa

SEQ ID NO: 22 - DNA, 21 bp, synthetic construct
  source 1..21: mol_type = other DNA
ggacggccttagcaagcatag

SEQ ID NO: 23 - DNA, 20 bp, synthetic construct
  source 1..20: mol_type = other DNA
gagaagttagggcaacgcag

SEQ ID NO: 24 - DNA, 20 bp, synthetic construct
  source 1..20: mol_type = other DNA
ccagtttccaaaaagtccca

SEQ ID NO: 25 - DNA, 20 bp, synthetic construct
  source 1..20: mol_type = other DNA
tttgtgccaagcaaagtctg

SEQ ID NO: 26 - DNA, 20 bp, synthetic construct
  source 1..20: mol_type = other DNA
ccagcattttgtcacattcg

SEQ ID NO: 27 - DNA, 20 bp, synthetic construct
  source 1..20: mol_type = other DNA
ggctgtaataatggcgagga

SEQ ID NO: 28 - DNA, 20 bp, synthetic construct
  source 1..20: mol_type = other DNA
tctccccatggaagatcaag

SEQ ID NO: 29 - DNA, 20 bp, synthetic construct
  source 1..20: mol_type = other DNA
gggttggataatgcaggaaa

SEQ ID NO: 30 - DNA, 20 bp, synthetic construct
  source 1..20: mol_type = other DNA
gcccgtacaaggcatagaaa

SEQ ID NO: 31 - DNA, 20 bp, synthetic construct
  source 1..20: mol_type = other DNA
gggaggaacatggaatttga

SEQ ID NO: 32 - DNA, 20 bp, synthetic construct
  source 1..20: mol_type = other DNA
cggagtcaatgagatacgcc

SEQ ID NO: 33 - DNA, 20 bp, synthetic construct
  source 1..20: mol_type = other DNA
cacagagcgatgcaggttta

SEQ ID NO: 34 - DNA, 20 bp, synthetic construct
  source 1..20: mol_type = other DNA
gtttaggcgtagcaccttcg

SEQ ID NO: 35 - DNA, 20 bp, synthetic construct
  source 1..20: mol_type = other DNA
gccgacatcttaagcgactc

SEQ ID NO: 36 - DNA, 20 bp, synthetic construct
  source 1..20: mol_type = other DNA
agtacgaggggatccttgct

SEQ ID NO: 37 - DNA, 20 bp, synthetic construct
  source 1..20: mol_type = other DNA
gatcgagctcttttcaacgg

SEQ ID NO: 38 - DNA, 20 bp, synthetic construct
  source 1..20: mol_type = other DNA
acgatggcaaaaacgaaaac

SEQ ID NO: 39 - DNA, 20 bp, synthetic construct
  source 1..20: mol_type = other DNA
ttccgtaatccagaggatgc

SEQ ID NO: 40 - DNA, 20 bp, synthetic construct
  source 1..20: mol_type = other DNA
gggcgtggaactgtatgtct

SEQ ID NO: 41 - DNA, 20 bp, synthetic construct
  source 1..20: mol_type = other DNA
aaaaatcggggttatttggg

SEQ ID NO: 42 - DNA, 20 bp, synthetic construct
  source 1..20: mol_type = other DNA
atgcatgtcacgttcgtgtt

SEQ ID NO: 43 - DNA, 20 bp, synthetic construct
  source 1..20: mol_type = other DNA
ggttttgggtgttggttgtc

SEQ ID NO: 44 - DNA, 20 bp, synthetic construct
  source 1..20: mol_type = other DNA
ctcctcttcttccagggctt

SEQ ID NO: 45 - DNA, 20 bp, synthetic construct
  source 1..20: mol_type = other DNA
gtggagcattgcagcaaata

SEQ ID NO: 46 - DNA, 20 bp, synthetic construct
  source 1..20: mol_type = other DNA
tggcagcctcttcactaggt

SEQ ID NO: 47 - DNA, 20 bp, synthetic construct
  source 1..20: mol_type = other DNA
aaaattaaaacgcaaccccc

SEQ ID NO: 48 - DNA, 20 bp, synthetic construct
  source 1..20: mol_type = other DNA
ttgggagagagggatgaatg

SEQ ID NO: 49 - DNA, 20 bp, synthetic construct
  source 1..20: mol_type = other DNA
tcttggaagatgggttacgc

SEQ ID NO: 50 - DNA, 20 bp, synthetic construct
  source 1..20: mol_type = other DNA
aatttttgggcttgttgtcg

SEQ ID NO: 51 - DNA, 20 bp, synthetic construct
  source 1..20: mol_type = other DNA
cggattctattggatgcgtt

SEQ ID NO: 52 - DNA, 20 bp, synthetic construct
  source 1..20: mol_type = other DNA
tcatgctgcaactcttgctt

SEQ ID NO: 53 - DNA, 20 bp, synthetic construct
  source 1..20: mol_type = other DNA
gggcttgaagagcaacagtc

SEQ ID NO: 54 - DNA, 20 bp, synthetic construct
  source 1..20: mol_type = other DNA
tggacatagctttgcagtcg

SEQ ID NO: 55 - DNA, 20 bp, synthetic construct
  source 1..20: mol_type = other DNA
agcagcagatcggcataagt

SEQ ID NO: 56 - DNA, 20 bp, synthetic construct
  source 1..20: mol_type = other DNA
acttcggacatggtgagacc

SEQ ID NO: 57 - DNA, 20 bp, synthetic construct
  source 1..20: mol_type = other DNA
cgacatcgctaacatcgcta

SEQ ID NO: 58 - DNA, 20 bp, synthetic construct
  source 1..20: mol_type = other DNA
cggactcgatgtagctctcc

SEQ ID NO: 59 - DNA, 20 bp, synthetic construct
  source 1..20: mol_type = other DNA
acatgttgcctttagacggg

SEQ ID NO: 60 - DNA, 20 bp, synthetic construct
  source 1..20: mol_type = other DNA
ctggcgtaaacgtgtgctaa

SEQ ID NO: 61 - DNA, 20 bp, synthetic construct
  source 1..20: mol_type = other DNA
aggattggtacgtggacagc

SEQ ID NO: 62 - DNA, 20 bp, synthetic construct
  source 1..20: mol_type = other DNA
caaggagatgacaccggact

SEQ ID NO: 63 - DNA, 20 bp, synthetic construct
  source 1..20: mol_type = other DNA
tgctcaactgcaaccgatag

SEQ ID NO: 64 - DNA, 20 bp, synthetic construct
  source 1..20: mol_type = other DNA
aactgcatgtaaggttcggg

SEQ ID NO: 65 - DNA, 20 bp, synthetic construct
  source 1..20: mol_type = other DNA
acgatgtgagcaagtcgtgt

SEQ ID NO: 66 - DNA, 20 bp, synthetic construct
  source 1..20: mol_type = other DNA
ggcagctgctactgaaatcc

SEQ ID NO: 67 - DNA, 20 bp, synthetic construct
  source 1..20: mol_type = other DNA
acggagggaagagattcgtt

SEQ ID NO: 68 - DNA, 20 bp, synthetic construct
  source 1..20: mol_type = other DNA
ttcctctggtcgctgacttt

SEQ ID NO: 69 - DNA, 20 bp, synthetic construct
  source 1..20: mol_type = other DNA
acctgataatgatttcgcgg

SEQ ID NO: 70 - DNA, 20 bp, synthetic construct
  source 1..20: mol_type = other DNA
gtttgagcggctggacttag

SEQ ID NO: 71 - DNA, 20 bp, synthetic construct
  source 1..20: mol_type = other DNA
caaagtgtacgccccatctt

SEQ ID NO: 72 - DNA, 20 bp, synthetic construct
  source 1..20: mol_type = other DNA
ccttgcttttccaacagctc

SEQ ID NO: 73 - DNA, 20 bp, synthetic construct
  source 1..20: mol_type = other DNA
ccaccagcgaggatagtgat

SEQ ID NO: 74 - DNA, 20 bp, synthetic construct
  source 1..20: mol_type = other DNA
tgcggcttctgttttctttt

SEQ ID NO: 75 - DNA, 20 bp, synthetic construct
  source 1..20: mol_type = other DNA
tcattttggagccttcatcc

SEQ ID NO: 76 - DNA, 20 bp, synthetic construct
  source 1..20: mol_type = other DNA
cctgcaggttcctgctttag

SEQ ID NO: 77 - DNA, 20 bp, synthetic construct
  source 1..20: mol_type = other DNA
aagacccgtgaagctgaaga

SEQ ID NO: 78 - DNA, 20 bp, synthetic construct
  source 1..20: mol_type = other DNA
ttgtgcctcaattccctttc

SEQ ID NO: 79 - DNA, 20 bp, synthetic construct
  source 1..20: mol_type = other DNA
caaagcgtcaatgtcgagaa

SEQ ID NO: 80 - DNA, 20 bp, synthetic construct
  source 1..20: mol_type = other DNA
tggtgccacttcctagatcc

SEQ ID NO: 81 - DNA, 20 bp, synthetic construct
  source 1..20: mol_type = other DNA
aagacccgtgaagctgaaga

SEQ ID NO: 82 - DNA, 20 bp, synthetic construct
  source 1..20: mol_type = other DNA
ttgtgcctcaattccctttc

SEQ ID NO: 83 - DNA, 20 bp, synthetic construct
  source 1..20: mol_type = other DNA
ttctagaacaggcggtgctt

SEQ ID NO: 84 - DNA, 20 bp, synthetic construct
  source 1..20: mol_type = other DNA
atggccacctcaacagaatc

SEQ ID NO: 85 - DNA, 20 bp, synthetic construct
  source 1..20: mol_type = other DNA
tgcatgtacaaagcagcaca

SEQ ID NO: 86 - DNA, 20 bp, synthetic construct
  source 1..20: mol_type = other DNA
gggatctagccttttctggg

SEQ ID NO: 87 - DNA, 20 bp, synthetic construct
  source 1..20: mol_type = other DNA
ggcctaatactgcctggtca

SEQ ID NO: 88 - DNA, 20 bp, synthetic construct
  source 1..20: mol_type = other DNA
atgccagaagcatccactct

SEQ ID NO: 89 - DNA, 20 bp, synthetic construct
  source 1..20: mol_type = other DNA
tatactcaacccgaggcgac

SEQ ID NO: 90 - DNA, 20 bp, synthetic construct
  source 1..20: mol_type = other DNA
gacgtgtaacggaagccatt

SEQ ID NO: 91 - DNA, 20 bp, synthetic construct
  source 1..20: mol_type = other DNA
cttcggttcagagagttgcc

SEQ ID NO: 92 - DNA, 20 bp, synthetic construct
  source 1..20: mol_type = other DNA
cgggtagcgtcattgttttt

SEQ ID NO: 93 - DNA, 20 bp, synthetic construct
  source 1..20: mol_type = other DNA
gggttgcgcttcaagccctt

SEQ ID NO: 94 - DNA, 22 bp, synthetic construct
  source 1..22: mol_type = other DNA
ccagaccttcctgttccagctc

SEQ ID NO: 95 - DNA, 20 bp, synthetic construct
  source 1..20: mol_type = other DNA
gtagccgaattgtacgggaa

SEQ ID NO: 96 - DNA, 20 bp, synthetic construct
  source 1..20: mol_type = other DNA
aattgcgatgtgccaacata

SEQ ID NO: 97 - DNA, 20 bp, synthetic construct
  source 1..20: mol_type = other DNA
gaaagggaaattgtccgtga

SEQ ID NO: 98 - DNA, 20 bp, synthetic construct
  source 1..20: mol_type = other DNA
agagggaagccaagatggat

SEQ ID NO: 99 - DNA, 20 bp, synthetic construct
  source 1..20: mol_type = other DNA
tcctcgccgacaaaataatc

SEQ ID NO: 100 - DNA, 20 bp, synthetic construct
  source 1..20: mol_type = other DNA
cctcggaatgatcgtgttct